(19) United States
(12) Patent Application Publication
(10) Pub. No.: US 2024/0164658 A1
(43) Pub. Date: May 23, 2024

(54) DEVICE AND A METHOD FOR DETERMINATION OF A MEASURE FOR THE HOMOGENEITY OF THE LUNG

(71) Applicant: SenTec AG, Therwil (CH)

(72) Inventor: Lisa KRUKEWITT, Rostock (DE)

(73) Assignee: SenTec AG, Therwil (CH)

(21) Appl. No.: 18/550,746

(22) PCT Filed: Mar. 15, 2022
(86) PCT No.: PCT/EP2022/056714

§ 371 Date: Sep. 15, 2023

(30) Foreign Application Priority Data
Mar. 18, 2021 (EP) 21163378.9

(51) Int. Cl. A61B 5/08; A61B 5/00; A61B 5/0536

(52) U.S. Cl. CPC A61B 5/0809; A61B 5/0536; A61B 5/7264

(57) ABSTRACT

The invention relates to a device and a method for determination of a measure for the homogeneity of the lung based on electrical impedance tomography (EIT) data as well as to a computer program. The device (100) comprises a data input unit (112), receiving the EIT data obtained by means of an electrical impedance tomography apparatus (110), wherein the data input unit (112) is configured to receive and provide EIT data from at least one region of at least one lung (2) of a living being over an observation period (ΔT). The device (100) also comprises a calculation and control unit (114) connected to the data input unit (112). The calculation and control unit (114) is configured to determine impedance values over the observation period (ΔT) for each pixel (1) of the at least one region of at least one lung (2), to determine an impedance amplitude value (ΔZ) and an end-expiratory impedance values (EELI) for each pixel (1) of at least one lung (2). The calculation and control unit (114) is further configured to associate the impedance amplitude values (ΔZ) and the end-expiratory impedance values (EELI) individually for each pixel (1), to determine data on the basis of the impedance amplitude values (ΔZ) and the associated end-expiratory impedance values (EELI) and to produce a control signal on basis of the data.

The invention relates to a device and a method for determination of a measure for the homogeneity of the lung based on electrical impedance tomography (EIT) data as well as to a computer program product.

Electrical impedance tomography (EIT) is a non-invasive imaging technique based on the application of current and measurement of voltage through electrodes attached to the body of a patient. EIT provides images of the distribution of conductivity, admitivity, impedivity, or resistivity, or changes thereof. In the following the measured values are called “impedance values” and said distribution is also referred to collectively as “electrical properties”. The image is called the EIT image. The image or sequences of images show differences in the electrical properties of various body tissues, bones, skin, body fluids and organs, particularly the lungs, which are useful for monitoring the patient's condition.

A typical EIT arrangement is shown in WO 2015/048917 A1. A set of electrodes is arranged on a belt, such that they are placed at a certain distance from each other around the chest of a patient, in electrical contact with the skin. An electrical current or voltage input signal is applied alternatingly between different or all possible pairs of electrodes. While the input signal is applied to one of the pairs of electrodes, the currents or voltages between the remaining electrodes may be measured. The measured electrical voltages of the body section may be reconstructed into electrical properties or changes of electrical properties by a reconstruction algorithm for use by a data processor to obtain a representation of the distribution impedance pedance values over a cross-section of the patient around which the electrode belt ring is placed. The electrical properties are displayed on a screen.

In the EIT process a number of impedance measurement values are recorded at for example 16 or 32 electrodes. From these impedance measurement values, the EIT image reconstruction algorithm may then produce two dimensional images comprising pixels representing properties of the lungs. The image may comprise 32×32 pixels which represent locations within and outside the lungs.

Information on electrical properties obtained by EIT can be projected into a cross-sectional image derived from an anatomical model in an anatomical context, showing for example contours representing the outer boundaries of the modelled organs within the electrode plane.

Contours of functional structures can be used to (automatically) cluster the pixels within such structures to form Regions Of Interest (ROI) that match with functionally meaningful anatomical structures such as the lungs. Signals of pixels falling within such ROIs can be identified and analysed separately for each one of the ROIs by automatic signal processing means and algorithms.

Up to 100 or even more tomographic EIT images per second may be reconstructed. These high time-resolution images reflect the regional electrical properties within the lung tissue, which are influenced by the respiratory and cardiac cycles. In contrast to CT scans EIT-images do not display morphological but functional information showing regional tidal ventilation, local lung recruitment, expiratory time constants or the distribution of lung perfusion. EIT also measures end expiratory lung impedance (EELI), a parameter which correlates with functional residual capacity (FRC) at the global level and/or with the end expiratory lung volume (EELV) on a regional or pixel level.

The capability of EIT provides information about ventilation at the level of individual pixels in the EIT image. However, currently no reliable conversion from impedance values to volume measurements exists, and therefore a direct calculation of volumes and volume changes from EIT data is not possible.

DE102017007224 discloses to determine properties and property changes of at least two regional areas of the lung on the basis of EIT data which represent regional changes in lung compliance or elasticity. The method does not provide a criterion at the level of the entire lung.

EP3725222A1 relates to a system for real-time determination of local stress on a lung during artificial respiration. A specific electrical impedance value at a specific point in time is assigned to a specific EIT pixel. A local tidal volume value z is determined as a difference between the end-inspiratory electrical impedance (ZINSP) and the end-expiratory electrical impedance (ZEXSP) of the specific EIT pixel. A preliminary strain value is determined by dividing the local tidal volume by the end-inspiratory electrical impedance. Preliminary strain values may be normalized to a reference strain value, for example at a predetermined PEEP value. Changes of the condition of the lung may be monitored for a specific patient, but a criterion of status of the lung as such may not be derived.

It is an object of the present invention to overcome the drawbacks of the prior art and in particular to provide a device and a method which allow to provide meaningful data concerning the lung in a simple and reliable way.

According to the invention these and other objects are solved with a device and a method for determining a measure for the homogeneity of the lung based on electrical impedance tomography (EIT) data according to the independent claims.

The ratio of change in lung volume dV during ventilation to the resting lung volume Vo or end-expiratory lung volume EELV is referred to as lung strain (Gattinoni et al. 2012, Stress and strain within the lung, Curr Opin Crit Care 18 (1): 42-7).

The device of the present invention is based on the finding that the determination, monitoring and analysis of strain on a pixel level by EIT can provide meaningful information even if no specific values of volume can be determined with EIT.

Lung strain resulting from increased respiratory drive during mechanical ventilation may inflict high energy loads on lung tissue thereby increasing the risk for patient self-inflicted lung injury (Brochard et al. 2017, Mechanical ventilation to minimize progression of lung injury in acute respiratory failure, Am J Respir Crit Care Med 195 (4): 438-42).

A global strain can be derived from the ratio of tidal volume VT divided by functional residual capacity FRC.

During mechanical ventilation, low total lung strain reduces the risk of ventilator induced lung injury (Protti et al. 2012, Lung stress and strain during mechanical ventilation: Any safe threshold?, Am J Respir Crit Care Med 185: 115). It is, however, largely unclear what the healthy boundaries of such a ratio dV/V₀ are and when strain becomes unphysiologically high (Gattinoni et al. 2012). In mechanically ventilated pigs, Protti et al. found no lung damage of strains below 1.5 and acute respiratory failure for strain values above 2.5, but a grey zone remains between these values (Protti et al. 2012; Gattinoni et al. 2012). However, Gattinoni points out that such high lung strain does not typically appear in mechanically ventilated patients and points to lung inhomogeneity as a possible primary cause of lung injury in mechanically ventilated patients (Gattinoni et al. 2012). So far, the common definition of strain as dV/V₀ at the level of the entire lung means that such inhomogeneities often go unnoticed as this would require regional lung strain measurements.

Therefore, when looking at strain as a cause of lung injury, especially in spontaneously breathing patients, the focus needs to be on the determination of regional strain and on identifying strain inhomogeneities. A homogeneous distribution of regional lung strain would mean that the ratio dV/V₀ of change in lung volume dV to the resting lung volume V₀ is approximately constant throughout the lung. Thus, dV needs to increase linearly with V₀ meaning that regions with high lung volume show larger ventilation.

US 20140316266 discloses to determine local lung strain, lung volume and changes in lung volume using ultrasound imaging.

The device according to the present invention comprises a data input unit.

The data input unit is designed for receiving EIT data obtained by means of an electrical impedance tomography apparatus comprising electrodes to be positioned on a patient.

The EIT data may be provided to the data input unit directly from the electro-impedance tomography apparatus or indirectly via data lines, signal lines or network connections.

The device may be associated with or comprise an electro-impedance tomography apparatus.

The data input unit is configured to receive and provide EIT data from at least one region of at least one lung of a living being over an observation period. The observation period typically lasts over a number of respiratory cycles. Preferably, EIT data are provided for a cross-section of at least one entire lung.

A calculation and control unit is connected to the data input unit. The calculation and control unit is configured to determine impedance values over the observation period for each pixel of the at least one region.

The calculation unit may assign EIT data measured in at least one observation plane to a region of interest such as the right and/or the left lung.

The impedance values are typically stored as a data set in a data memory or data storage area assigned to the calculation and control unit and are kept available for further data processing in the data memory or data storage area.

The calculation and control unit is further configured to determine an impedance amplitude value for each pixel of at least one lung.

The amplitude is indicative of a difference between a maximal impedance value and a minimal impedance value within a respiratory cycle. The amplitude may be defined based on absolute maxima and minima within a cycle, but also on averages or on the basis of successive maxima and minima. The impedance amplitude value may be determined by a mean impedance difference over the observation period or by the maximal difference occurring over the observation period.

The calculation and control unit is further configured to determine an end-expiratory impedance value for each pixel of at least one lung. The end-expiratory impedance values may be calculated as the average minimum of the impedance values over the observation period for each pixel.

The calculation and control unit is further configured to determine data on the basis of the impedance amplitude values and the associated end-expiratory impedance values individually for each pixel.

The data may be used for a mapping of the impedance amplitude values and/or the end-expiratory impedance values to each pixel.

The data may provide a representation of a local distribution of measured values.

The data may be used to provide a graphical representation showing the relation between the change in impedance and the end-expiratory lung impedance for each pixel. For an unaffected lung the impedance amplitude values and the end-expiratory impedance values are assumed to be correlated to a certain extent.

Based on the determined data, the calculation and control unit may provide at least one number or at least a range of numbers which are characteristic for assignment relationship between the impedance amplitude values and the associated end-expiratory impedance values for each pixel.

The calculation and control unit is further configured to generate a control signal on basis of the data. The control signal may be used for forwarding, storing and/or displaying the data or information derived from the data.

The data may be analysed by a skilled person, who may draw conclusions from the pattern of the data. Data may also be used for automatic control of medical devices.

In particular the calculation and control unit is further configured to determine if the data fulfil a predetermined criterion. The data may be e.g. compared with a predetermined criterion, such as a predetermined data pattern, a predetermined threshold number or a predetermined range. An inhomogeneity indicator may be generated if the criterion is not fulfilled.

The criterion may be predetermined on the basis of a control group of patients. The control group may consist of persons having unaffected lungs.

Impedance amplitude values and associated end-expiratory impedance values may be measured or determined for the control group.

The criterion may be derived from a local distribution of impedance amplitude values, associated end-expiratory impedance values and/or of data deduced from impedance amplitude values and associated end-expiratory impedance values over pixels of the lung.

The criterion may be derived from a distribution of impedance amplitude values over associated end-expiratory impedance values.

The comparison may provide at least one difference between data obtained for a patient and the criterion and/or a ratio between data obtained for a patient and the criterion.

In EIT, determining absolute values of impedance and from this, lung volumes is difficult, especially without calibration. Therefore, although distributions and changes of volume can be quantified, intra-patient comparability remains difficult. Although the changes of volume and the resting lung volume relate to the impedance amplitude values and the end-expiratory lung impedance respectively, these measurements cannot be converted to volume measurements. Especially values for end-expiratory impedance values may vary greatly in EIT recordings of even the same patient, which may cause vastly different strain values if calculated as a simple ratio of the impedance amplitude values and the end-expiratory lung impedance.

Therefore, absolute values of the ratio of the impedance amplitude values and the end-expiratory lung impedance may vary between measurements of patients with similar lung strain or even between successive measurements of the same patient.

Thus, instead of focusing on absolute strain values, the data derived from associated impedance amplitude values and end-expiratory impedance values and in particular from a local distribution of impedance amplitude values and end-expiratory impedance values within the lung and/or of a statistical distribution of values derived from impedance amplitude values and end-expiratory impedance values can be used as a measure of the in-homogeneity of the lung.

This makes the method less susceptible to distortion by single outliers.

In healthy lungs, strain is low and assumed to be approximately constant throughout the lung with high changes in lung volume occurring in areas of high resting lung volume.

To identify regions with higher strain than in the surrounding tissue, no absolute values of the change in lung volume or the resting lung volume are needed according to the present invention.

Rather, an analysis can be based on a pixel-wise comparison of parameters that can be measured by EIT.

The calculation and control unit may be in particular configured to perform a function fit of the impedance amplitude values in dependency of the end-expiratory impedance values for at least one lung and to obtain a fitted function, in particular to obtain parameters characterizing the fitted function.

As regions with high lung volume show larger ventilation, and larger impedance variations, for example a linear relation between the impedance amplitude values and the end-expiratory impedance values may be assumed.

For example a linear regression may be performed, for example to obtain a linear function ΔZ_{ideal}=αEELI+β for at least one lung.

The fitted values represent a constant strain in each lung.

Even though impedance changes measured by EIT depend on a variety of patient-specific factors, such as thorax shape, making a calculation of tidal volume from EIT at least difficult if not almost impossible without calibration, it has been found that the impedance amplitude value can be assumed to increase linearly with the change in lung volume.

EIT provides a distribution of impedance changes proportional to regional tidal volumes. In the same manner, it may be assumed that end-expiratory lung impedance increases linearly with end-expiratory lung volume. It may be assumed that the distribution of end-expiratory lung impedance is proportional to the regional resting lung volume.

From the assumption of a constant ratio of change in lung volume and resting lung volume, a linear relationship between the impedance amplitude values and the end-expiratory impedance values can be assumed.

Depending on the reconstruction of the EIT image areas with an end-expiratory impedance close to zero do not show ventilation in healthy individuals since such high conductivities are not typical for healthy lung tissue. Therefore the offset β can be assumed zero and the ratio between the impedance amplitude values and the end-expiratory impedance values can be assumed to be a constant.

To identify regions with higher strain than in surrounding lung tissue absolute values for the regional distribution of changes in lung volume and resting lung volume are not needed. Rather, only information about the deviation of these parameters from a predefined, e.g. linear relationship at the regional level is needed.

As derived above, the distribution of tidal volume and lung volume within the lung relates to values that can be measured by EIT: tidal variation of impedance and end-expiratory impedance of each pixel. Therefore, EIT could provide real-time measurements of regional distribution of lung strain.

The calculation and control unit may further be configured to determine a distribution of impedance amplitude values with respect to the fitted values and to produce a control signal on basis of the data. For example, impedance amplitude values and fitted values may be assigned to a respective end-expiratory impedance value for each pixel. Alternatively or additionally, impedance amplitude values and fitted values may be assigned to the respective pixels. The distribution of impedance amplitude values may be compared with the distribution of fitted values.

The distribution of the impedance amplitude values may provide a measure for the status of the lung. When the distribution is sufficiently close to the fitted values which represent an ideal status, it can be assumed that the lung has sufficient homogeneity. The larger the deviation of the distribution, the larger is the likelihood of inhomogeneity which may cause a malfunction.

The device for determination of a measure for the homogeneity of the lung and/or the data input unit and/or the calculation and control unit may be part of stand-alone EIT device or may be part of an external device, such as a personal computer, a patient monitor or a data processing system of a hospital.

The device for determination of a measure for the homogeneity of the lung may interact with another medical device, such as a ventilator or anaesthetic apparatus, and may form a medical technology system.

In a beneficial embodiment of the device the calculation and control unit is configured to determine for each pixel a deviation value representing the deviation of the impedance amplitude value from the fitted value.

The deviation may for example be determined as the ratio between the impedance amplitude value and the fitted value at the same end-expiratory impedance value.

The deviation may alternatively be determined by the difference between the impedance amplitude value and the fitted value at the same end-expiratory impedance value, the square of the difference or by the logarithm of the ratio between the impedance amplitude value and the fitted value at the same end-expiratory impedance value (log (ΔZ/ΔZ_{ideal})).

The deviation may be a measure of the lung inhomogeneity.

The closer the ratio between the impedance amplitude value and the fitted value is to 1.0, the closer are the respective impedance amplitude values to the fitted values. Hence, the more pixels have a ratio close to 1.0, the greater is the likelihood of an unaffected lung. A great homogeneity is suggestive of a healthy lung. A narrow distribution of ratio values around 1.0 reflects activity of healthy lung tissue during normal breathing.

Alternatively or additionally, the coefficient of determination R² may be used as a criterion for the lung inhomogeneity.

The calculation and control unit may be configured to determine a histogram showing the number of pixels with deviation values in certain intervals for each of the intervals for all pixels of at least one lung.

The distribution of the histogram may give an indication about the status of the lung. The longer the tail on the right side of the value 1.0, the greater the inhomogeneity of the lung and the greater the likelihood of a damage.

The calculation and control unit may be configured to determine an amount of pixels for which the deviation value is larger than a predetermined threshold and/or outside a predetermined region, in particular a predefined region around the fit function, such as a confidence interval.

The amount of pixels may be given by the absolute number or by a percentage.

A threshold may be defined by a cut-off for values which are deemed to be representative for a pathologic condition.

A threshold may be defined by a value, below which a certain percentage (for example 95%) of deviations of all pixels for a reference patient or for a reference group of, in particular healthy, patients is present.

The borders of the confidence interval or the number of pixels above the threshold may be used as a measure for the homogeneity or the inhomogeneity of the lung.

In an advantageous embodiment the device comprises an output unit and the output unit is configured to use the control signal to provide or output an output signal for displaying a representation of the data.

The output unit may be part of an EIT device or may be part of an external device.

The output unit may display a graphical representation associating the impedance amplitude values and/or fitted amplitude values with the end-expiratory impedance values (EELI) for each pixel, for example a two dimensional graph showing impedance amplitude values ΔZ and fitted values in dependency of end-expiratory impedance values.

The output unit may alternatively or additionally display a regional distribution of end-expiratory impedance values, impedance amplitude values and/or fitted values over at least one lung.

The output unit may alternatively or additionally display a histogram of numbers of pixels being associated with a deviation value within a certain interval, in particular an interval of ratios of impedance amplitude values and fitted amplitude values.

The output unit may alternatively or additionally display an amount of pixels for which a deviation value is larger than a predetermined threshold and/or which are outside a predetermined region.

Without calibration, the absolute values of the impedance amplitude values and the end-expiratory impedance values do not correspond to a certain volume in ml. As there is primarily an interest in the pattern of deviation value distribution, the impedance amplitude values may be normalized over all pixels for at least one lung to obtain a normalized impedance amplitude values for all pixels of at least one lung.

Normalization may be achieved by dividing the impedance amplitude values by the maximum value, in particular for each lung separately.

Alternatively or additionally the end-expiratory impedance values may be normalized over all pixels for at least one lung to obtain a normalized end-expiratory impedance values for all pixels of at least one lung.

The normalized values range up to 1.

In a preferred embodiment the device is configured to obtain, to determine and/or to display reference data, for example EIT data of reference patients, a reference distribution of impedance amplitude values, fitted values and/or a reference histogram.

Preferably, reference data can be determined on the basis of electrical impedance tomography data of reference patients. Reference patients may be patients with a healthy lung. Reference data may be obtained by determining average values for a number of reference patients.

The calculation and control unit may be configured to independently determine data for each of the lungs.

A method for determination of difference parameters based on electrical impedance tomography (EIT) data according to the invention, preferably performed on a device as described above, comprises the following steps.

EIT data are provided from at least one region of at least one lung of a living being over an observation period.

Impedance values are determined over the observation period for each pixel of the at least one region of at least one lung.

An impedance amplitude value and an end-expiratory impedance value are determined for each pixel of at least one lung.

The impedance amplitude value (ΔZ) is associated with the end-expiratory impedance values (EELI) for each pixel. Data are determined on the basis of this information.

A control signal on the basis of the data is generated.

In particular, a calculation and control unit analyses if the data fulfil a predetermined criterion. The data may be compared with a predetermined criterion, such as a predetermined data pattern, a predetermined number or a predetermined range.

A function fit may be performed, for example a linear regression, of the impedance amplitude values in dependency of the end-expiratory impedance values for at least one lung. A fitted function may be obtained, for example a linear function ΔZ_{ideal}=αEELI+β, for at least one lung.

Data representative of a distribution of impedance amplitude values with respect to the fitted values may be determined. A control signal on the basis of the data may be produced.

An EIT-based statistical lung strain distribution may be defined as a deviation from a linear relationship between impedance amplitude values and end-expiratory impedance values.

This can be determined on the level of individual pixels.

Assuming that all pixels corresponding to areas having a healthy ventilation have a linear relationship between impedance amplitude values and end-expiratory impedance values, a plot of impedance amplitude values as a function of end-expiratory impedance values would lie on a straight line for all pixels.

If a larger part of the lung shows healthy ventilation, an ideal ratio between impedance amplitude values and end-expiratory impedance values can be determined from a linear regression of all pixels. Impedance amplitude values lying on this fitted function indicate a constant strain distribution within the lung.

For each pixel a deviation value representing the deviation of the impedance amplitude value from the fitted value may be determined.

To quantify the deviation of each pixel from its ideal strain, a ratio between the measured impedance amplitude value and the fitted function for the same end-expiratory impedance value may be calculated. This may result in a number of deviation values which have a certain statistical distribution.

In a healthy lung the median of the values is close to 1.0. Since the impedance amplitude value is always larger than zero, so is the deviation.

An amount of pixels may be determined for which the deviation value is larger than a predetermined threshold and/or outside a predetermined region.

An output signal may be provided or output for displaying the data. In particular an output signal showing a two dimensional graph of impedance amplitude values and a fitted function in dependency of end expiratory values may be displayed. Additionally or alternatively at least one histogram showing amounts of deviation values may be displayed.

A cut-off may be defined based on the measurements in healthy volunteers. This threshold is necessary since even in healthy lungs, some fluctuations in impedance amplitude value and end-expiratory impedance values will be observed that will lead to deviation values being unequal 1.0.

Data for each of the lungs and/or reference data may be determined and/or displayed independently.

The data determined on the basis of the impedance amplitude values and the associated end-expiratory impedance values for each pixel may be compared with a respective distribution of reference amplitude values and/or deviation values obtained from healthy patients. Depending on the comparison, conclusions about a lung disease or lung malfunction may be drawn, even in an early stage. The device and the method according to the invention may therefore provide a diagnostic tool.

The object of the invention is also solved by a computer program product directly loadable into the internal memory of a computer, in particular into a calculation and control unit of a device as described above and/or an EIT device, wherein the computer program product comprises software code portions for performing the steps of a method as described above when said product is run on a computer.

The invention will now be described with reference to preferred embodiments and the drawings, which show:

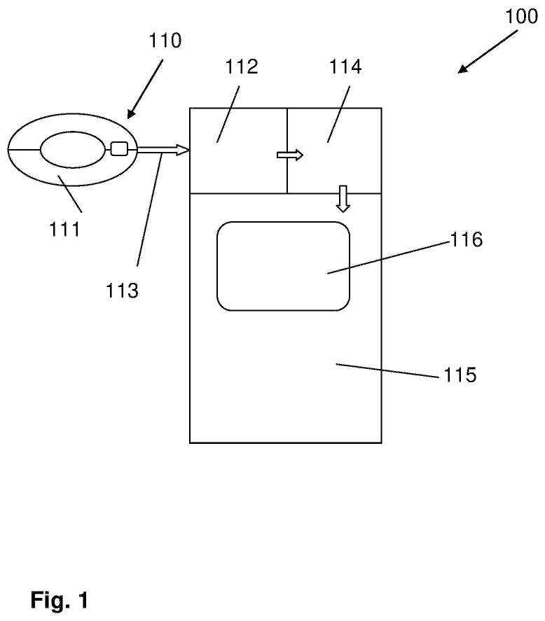

FIG. 1 a schematic presentation of a device for determination of a measure for the homogeneity of the lung based on electrical impedance tomography (EIT) data;

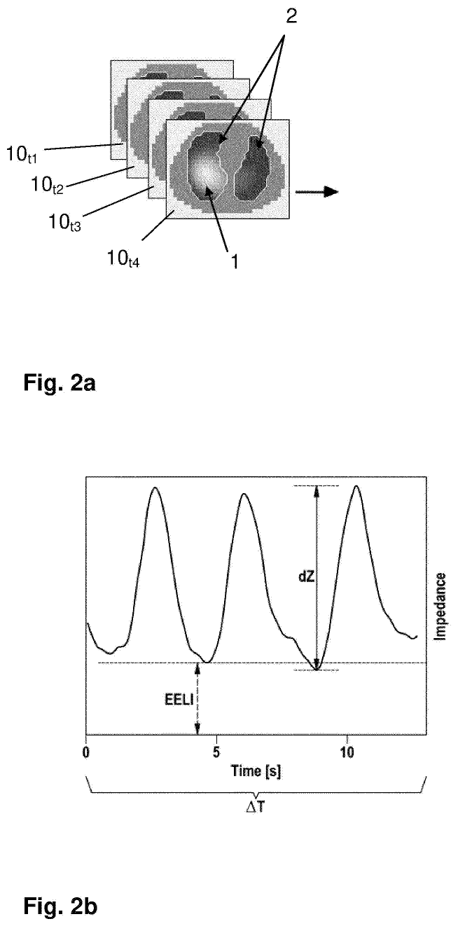

FIG. 2a a schematic representation of EIT images during an observation period for a first exemplary patient;

FIG. 2b an example for a change of the measured impedances for one pixel during an observation period;

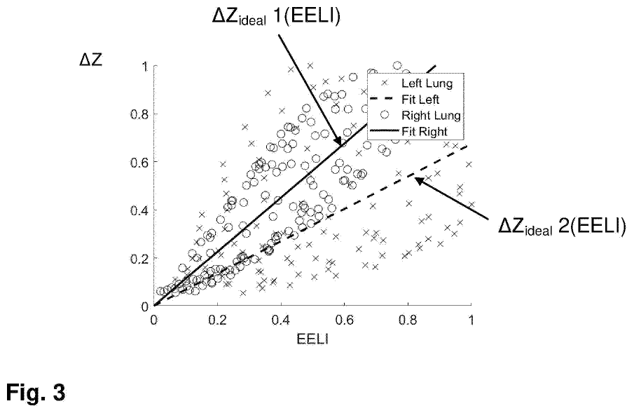

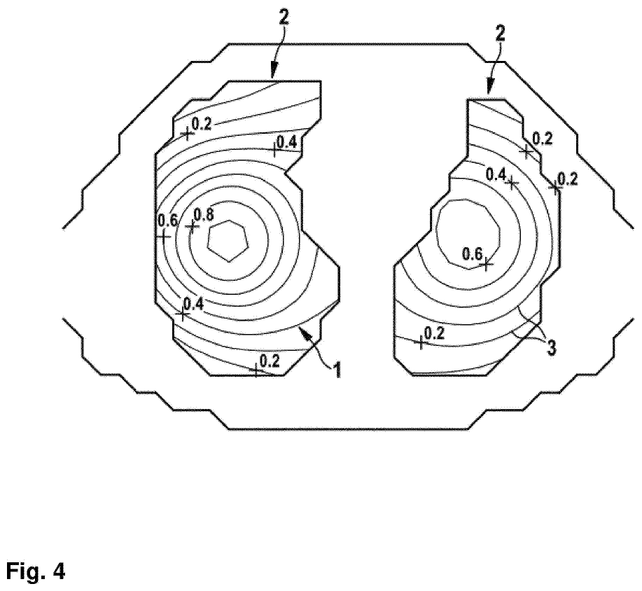

FIG. 3 a graphical representation of impedance amplitude values in dependence of respective end-expiratory impedance values for the first exemplary patient;

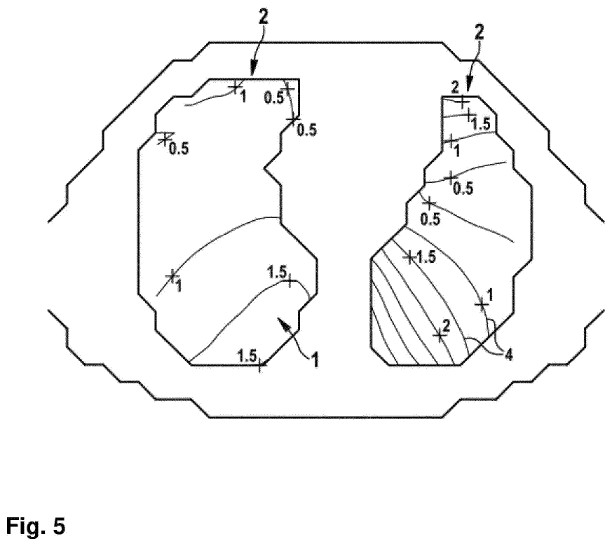

FIG. 4 a graphical representation of fitted amplitude values for each pixel of the lungs of the first exemplary patient;

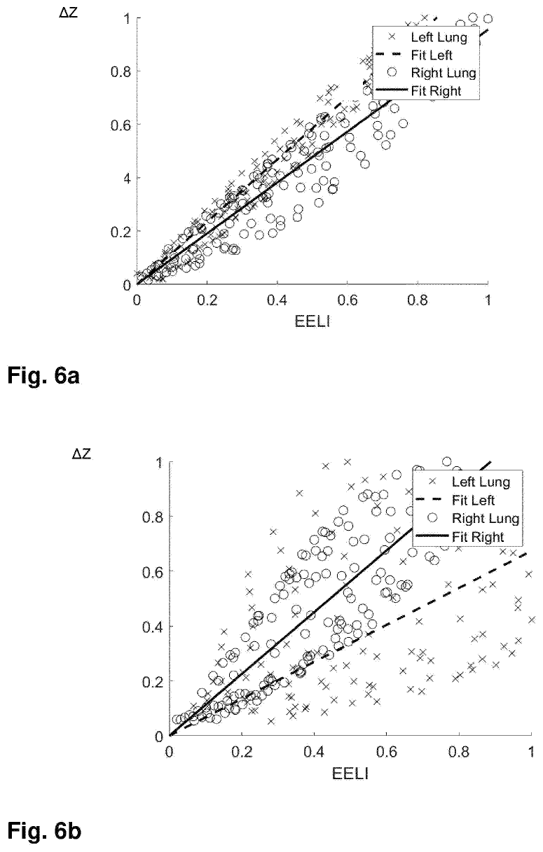

FIG. 5 a graphical representation of deviation values for each pixel of the lungs of the first exemplary patient; FIG. 6a a graphical representation of impedance amplitude values in dependence of respective end-expiratory impedance values of a reference patient;

FIG. 6b a graphical representation of impedance amplitude values in dependence of respective end-expiratory impedance values of a COVID 19 patient;

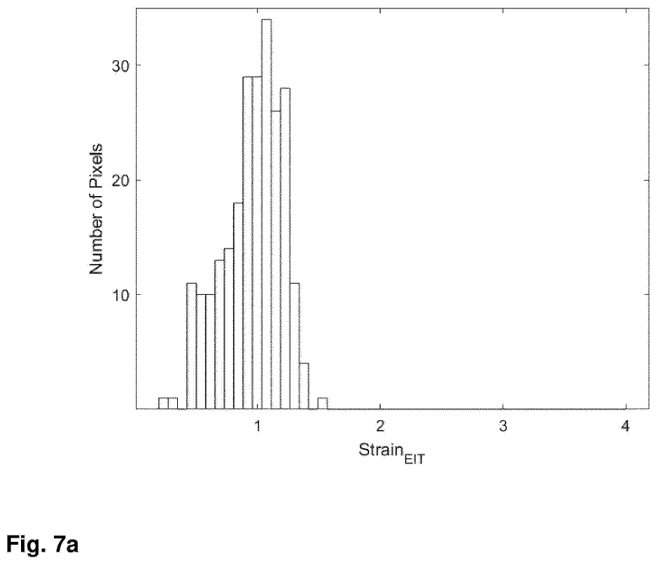

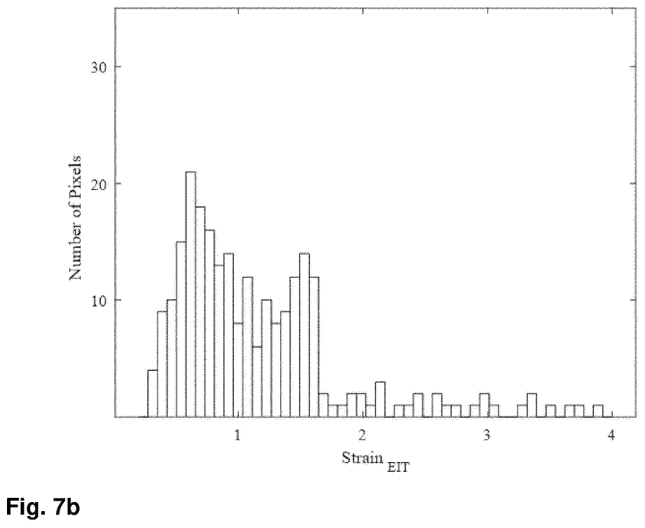

FIG. 7a a graphical representation of deviation values of the reference patient;

FIG. 7b a graphical representation of deviation values of the COVID 19 patient;

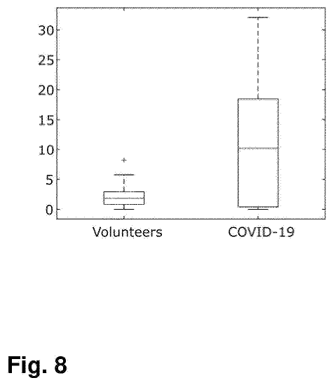

FIG. 8 a graphical representation of percentages of pixels with a deviation value larger than a threshold of a group of COVID 19 patients in comparison with reference patients.

FIG. 1 shows a schematic presentation of an example for a device 100 for determination of a measure for the homogeneity of the lung based on electrical impedance tomography (EIT) data.

The device 100 comprises an EIT apparatus 110 with a belt 111 with electrodes not explicitly shown in the figures. EIT data obtained by the EIT apparatus 110 are provided to a data input unit 112 via a data line 113. A calculation and control unit 114 is connected to the data input unit 112. The calculation and control unit 114 processes EIT data, determines various values representing a measure for the homogeneity of the lung and provides output signals as described below.

These output signals may be provided to an output unit 115, which comprises a display unit 116 for displaying the determined values.

EIT measurements were recorded with the Sentec BB2 (Sentec AG, EIT branch, Landquart, Switzerland) using a textile electrode belt with 32 electrodes. Impedance tomography data was recorded at a sampling rate of 47.68 Hz. Patient-specific ventilation images of breathing-induced impedance changes were calculated in relation to a reference measurement using the manufacturer's imaging algorithm. All calculations were done offline using Matlab R2018b (The MathWorks, Natick, Massachusetts). The images of 32 by 32 pixels created by the manufacturer's imaging algorithm were used to calculate strain images.

FIG. 2a shows a schematic representation of EIT images 10ₜ₁, 10ₜ₂, 10ₜ₃, 10ₜ₄ for different points of time during an observation period ΔT for a first exemplary patient. The EIT image is based on impedance values at pixels 1 of each of the lungs 2 and shows impedance values measured for each pixel in an observation plane defined by the position of the belt 111 on the patient. Each pixel of the EIT mage is coloured according to the impedance value of the pixel.

FIG. 2b shows an example for a change of the measured impedance values for one pixel 1 during an observation period ΔT.

An impedance amplitude value ΔZ is determined as the maximal difference between a maximum and a subsequent minimum of the measured impedance during the observation period ΔT.

An end-expiratory impedance value EELI may be determined as the average minimum value of the impedance during the observation period ΔT.

For each pixel 1 of both lungs 2 the impedance amplitude value ΔZ and the end-expiratory impedance values EELI is determined.

FIG. 3 shows a graphical representation of impedance amplitude values ΔZ in dependence of respective end-expiratory impedance values EELI for the first exemplary patient.

Since there is primarily an interest in the pattern, impedance amplitude values ΔZ and end-expiratory impedance values EELI were normalized to range up to 1.0. The impedance amplitude values ΔZ and the end-expiratory impedance values EELI were divided by their respective maxima.

The impedance amplitude values ΔZ were normalized for both lungs separately, dividing ΔZ for each lung 2 by the maximum value in that lung 2.

The normalized impedance amplitude values ΔZ for each pixel are plotted against their respective normalized end-expiratory impedance values EELI.

A function fit, in this case a linear regression ΔZ=α·EELI, is performed for both lungs separately to obtain fitted functions ΔZ_{ideal}1 (EELI) and ΔZ_{ideal}2 (EELI) for each of the both lungs 2. The values ΔZ_{ideal}1, ΔZ_{ideal}2 are the values that would correspond to the specific end-expiratory impedance values if there was a constant strain in each lung.

Similarly, a function fit ΔZ=α·EELI+β may be performed, wherein EELI could be determined/rescaled in a way that eliminates the offset β. The linear relationship between ΔZ and EELI mirrors a linear relationship between change in lung volume dV and the resting lung volume V₀.

ΔZ_{ideal}1, ΔZ_{ideal}2 for each pixel of the respective lungs 2 of the exemplary patient is shown in FIG. 4. Each pixel 1 of the observation plane assigned to one of the lungs 2 is coloured according to the value ΔZ_{ideal}1 or ΔZ_{ideal}2 for the respective value EELI of the respective pixel.

FIG. 4 shows a graphical representation of fitted amplitude values for each pixel of the lungs of the first exemplary patient, wherein equal values of the amplitude values are shown as level lines 3.

It can be seen that a linear fit represents a reasonable model of a normal behaviour of the lungs 2.

Values with large ideal amplitudes ΔZ_{ideal} are located at pixels in the centre of the lung 2, whereas smaller values can be found in the boundary regions of the lungs 2.

For each pixel a deviation value Strain_{EIT} may be determined representing the deviation of the impedance amplitude value ΔZ from the fitted value ΔZ_{ideal}.

In this example, the deviation value Strain_{EIT} is obtained by the ratio ΔZ/ΔZ_{ideal} between the impedance amplitude value ΔZ and the fitted value ΔZ_{ideal} at the same end-expiratory impedance value EELI for each pixel 1 of each lung 2.

FIG. 5 shows a graphical representation of the regional distribution of the deviation values Strain_{EIT}, ΔZ/ΔZ_{ideal}1 and ΔZ/ΔZ_{ideal}2, for the first exemplary patient. Each pixel 1 of the observation plane belonging to one of the lungs 2 is assigned to the value ΔZ/ΔZ_{ideal}1 or ΔZ/ΔZ_{ideal}2 for the end-expiratory value EELI of the respective pixel. Equal values of the respective deviation values Strain_{EIT}, ΔZ/ΔZ_{ideal}1 and ΔZ/ΔZ_{ideal}2, are shown as level lines 4 for each of the lungs 2.

As can be seen in comparison with FIG. 4, the deviation values are distributed differently from the fitted values ΔZ_{ideal}.

Regions of the lungs may be identified, where the deviation values ΔZ/ΔZ_{ideal}1 deviate more or less from an ideal and undisturbed deviation value of 1.0. These regions may be considered to have a disturbed lung strain.

Hence, areas with high strain within the lung as well as patients with unphysiological lung inhomogeneity can be identified.

FIG. 6a shows a graphical representation of normalized impedance amplitude values ΔZ in dependence of respective normalized end-expiratory impedance values EELI of a reference patient, whereas FIG. 6b shows a graphical representation of normalized impedance amplitude values ΔZ in dependence of respective end-expiratory impedance values EELI of a COVID 19 patient.

Both representations show measured pixel values and fit functions for both lungs separately.

Impedance amplitude values ΔZ and respective end-expiratory impedance values EELI of the reference patient were obtained by an EIT measurement at a healthy volunteer.

For COVID-19 patients, two consecutive EIT measurements were performed 3 days apart. For healthy volunteers, only one measurement was recorded. All measurements may be recorded in sitting, supine, left and right lateral position. Examples shown in this application are based only from EIT recordings in the supine position.

Lung strain resulting from increased respiratory drive may inflict high energy loads on lung tissue thereby increasing the risk for patient self-inflicted lung injury. Such high lung strain may be contributing to respiratory failure in COVID-19 patients. Respiratory failure due to COVID-19 pneumonia may progress rapidly to an acute respiratory distress syndrome (ARDS) like clinical picture with an extremely inhomogeneously damaged lung. These patients often develop, even in the early phase, a severe hypoxemia and a pathologic respiratory drive with high respiratory load. During the course of the disease this increased respiratory drive, which induces high tidal strain and energy loads on the vulnerable lung tissue, increases the risk for Patient Self-Inflicted Lung Injury.

Even in a healthy lung, strain is not zero. Instead, the volume change during ventilation in a region is compatible with the total lung volume in that region resulting in a physiological level of strain. Furthermore, even in a healthy lung, some pixels will deviate from the linear relationship. Therefore a typical distribution of these values needs to be defined from measurements in healthy volunteers.

Accordingly, impedance amplitude values ΔZ are not exactly on the fitted linear line.

In a study including ten healthy volunteers aged 32±8 years and ten COVID-19 patients aged 55±21 years, overall a good correlation, with a mean R² value of 0.80 for COVID 19 patients and of 0.92 for healthy volunteers was found. R²-can be calculated by R²=1−sum(ΔZ−ΔZ_{ideal})²/sum(ΔZ−mean(ΔZ))². The lowest R² values were 0.77 in volunteers and 0.29 in patients.

Correlation itself, as indicated by the coefficient of determination R² may be considered as a measure of strain and/or a measure for inhomogeneity. However, information about which pixels show the right ratio ΔZ/EELI of impedance amplitude value ΔZ and end-expiratory impedance values EELI becomes less clear the fewer healthy lung pixels there are. Therefore, information about the regional distribution of strain should be handled with caution in cases of low R².

However, as can be seen in FIG. 6b the variation of impedance amplitude value with respect to the fitted function is way greater for the COVID-19 patient than for the healthy reference patient.

Thus, the distribution of normalized impedance amplitude values ΔZ in dependence of respective normalized end-expiratory impedance values EELI may be considered to be a measure of the inhomogeneity of the lung.

FIG. 7a shows a graphical representation of deviation values Strain_{EIT} of the reference patient and FIG. 7b shows a graphical representation of deviation values Strain_{EIT} of the COVID-19 patient.

The deviation values Strain_{EIT} were calculated as the ratio of the impedance amplitude values ΔZ and the value of the fitted function ΔZ_{ideal} at the same end-expiratory impedance values EELI.

The mean of these ratios is always 1.0, and in a healthy lung most pixels have deviation values Strain_{EIT} close to 1.0.

Physiologically, the values that are too high are more critical for patients. Alternatively, for example log (ΔZ/ΔZ_{ideal}) could be determined for all pixels. This consideration provides tails on both sides of the ideal value and allows to set a threshold on both sides of the ideal value. Not only values of ΔZ/ΔZ_{ideal} which are too large, but also values of ΔZ/ΔZ_{ideal} which are too small can be taken into account.

FIGS. 7a and 7b show histograms of numbers of pixels with a deviation value Strain_{ETI} being in a respective one of equidistant intervals between two deviation values. In this case the intervals have the lengths of 1/10.

As can be seen in FIG. 7a for the healthy reference patient as expected there is a maximum at 1 and most of the pixels are at least close to 1.

For the COVID-19 patient the histogram shows a long tail meaning that many pixels have a large deviation value Strain_{EIT}.

A cutoff may be defined based on the measurements in a sample group of healthy volunteers. Assuming that in healthy lung tissue only a few outlier pixels will show unphysiologically large strain, the ratio of impedance amplitude value ΔZ and fitted function values ΔZ_{ideal}, for which 97.5% of all pixels in healthy volunteers have a lower ratio, may be determined as the cutoff for healthy lung strain.

In images of the regional strain EIT distribution (see FIG. 5), pixels with values above this threshold show areas of high lung strain.

Furthermore, the number of pixels displaying such high deviation values can be an indicator for total lung strain. Hence, the sum of those pixels can be considered as an indicator for the total strain.

In patients of said study, an average of 21 out of 239 pixels in the lung region showed a strain value larger than 2.11, while this was only the case for an average of 6 pixels out of 242 in the lung region in the healthy volunteers.

FIG. 8 shows a graphical representation in the form of box-plots for percentages of pixels with a deviation value larger than a threshold of a group of COVID 19 patients of the study in comparison with reference patients of the study.

The difference in this number between patients and volunteers is conserved to be statistically significant at a p-value calculated using a two-sample test of 0.028.

p is the probability that the strain values for patients and volunteers come from independent random samples from normal distributions with equal means and equal but unknown variances (null hypothesis). A test statistic t is calculated and p is the probability of observing a test statistic as extreme as, or more extreme than, the observed value under the null hypothesis. The calculation is done with statistical software which uses numerical methods. Here, Matlabs's ttest2 function was used.

The median percentage of pixels larger than 2.11 was 1.8% for healthy volunteers and 10.2% for COVID-19 patients.

1-18. (canceled)

19. A device for determination of a measure for the homogeneity of the lung based on electrical impedance tomography (EIT) data, the device comprising:
a data input unit, receiving the EIT data obtained by an electrical impedance tomography apparatus, wherein the data input unit is configured to receive and provide EIT data from at least one region of at least one lung of a living being over an observation period (ΔT);
a calculation and control unit connected to the data input unit, wherein the calculation and control unit is configured
to determine impedance values over the observation period (ΔT) for each pixel of the at least one region of at least one lung;
to determine an impedance amplitude value (ΔZ) for each pixel of at least one lung;
to determine an end-expiratory impedance value (EELI) for each pixel of at least one lung;
to associate the impedance amplitude values (ΔZ) and the end-expiratory impedance values (EELI) individually for each pixel;
to determine data on the basis of the impedance amplitude values (ΔZ) and the associated end-expiratory impedance values (EELI), and
to generate a control signal on basis of the data.

20. The device according to claim 19, wherein the calculation and control unit is configured to compare the data with a predetermined criterion.

21. The device according to claim 19, wherein the calculation and control unit is configured to compare the data with a criterion, which criterion has been predetermined on the basis of a control group of patients.

22. The device according to claim 19, wherein the calculation and control unit is configured to perform a function fit of the impedance amplitude values (ΔZ) in dependency of the end-expiratory impedance values (EELI) for at least one lung to obtain a fitted function (ΔZ_{ideal}(EELI)) for at least one lung.

23. A device according to claim 22, wherein the fitted function (ΔZ_{ideal}(EELI)) is a linear function ΔZ_{ideal}=αEELI+β.

24. The device according to claim 22, wherein the calculation and control unit is configured to determine for each pixel a deviation value (Strain_{EIT}) representing the deviation of the impedance amplitude value ΔZ from the fitted value (ΔZ_{ideal}).

25. The device according to claim 24, wherein the deviation value (Strain_{EIT}) is the ratio (ΔZ/ΔZ_{ideal}) between the impedance amplitude value (ΔZ) and the fitted value (ΔZ_{ideal}) at the same end-expiratory impedance value (EELI).

26. The device according to claim 24, wherein the calculation and control unit is configured to determine a histogram showing the number of pixels with deviation values (Strain_{EIT}) in certain intervals for each of the intervals for all pixels of at least one lung.

27. The device according to claim 24, wherein the calculation and control unit is configured to determine an amount of pixels for which the deviation value (Strain_{EIT}) is larger than a predetermined threshold and/or for which the deviation value (Strain_{EIT}) is outside a predetermined region.

28. The device according to claim 19, wherein the device comprises an output unit and the output unit is configured to use the control signal to provide or output an output signal for displaying a representation of the data.

29. The device according to claim 28, wherein the output unit is configured to display at least one of
a graph associating the impedance amplitude values (ΔZ) and/or fitted amplitude values (ΔZ_{ideal}) with the end-expiratory impedance values (EELI) for each pixel,
a regional distribution of end-expiratory impedance values (EELI), impedance amplitude values (ΔZ) and/or fitted values (ΔZ_{ideal}) over at least one lung,
a histogram of numbers of pixels being associated with a deviation value (Strain_{EIT}) within a certain interval,
a histogram of numbers of pixels being associated with a deviation value (Strain_{EIT}) within an interval of ratios of impedance amplitude values (ΔZ) and fitted amplitude values (ΔZ_{ideal}), and
a diagram showing an amount of pixels (1) for which a deviation value (Strain_{EIT}) is larger than a predetermined threshold and/or for which a deviation value (Strain_{EIT}) is outside a predetermined region.

30. The device according to claim 19, wherein the calculation and control unit is configured to normalize the impedance amplitude values (ΔZ) over all pixels for at least one lung and to obtain a normalized amplitude value (ΔZ) for all pixels of at least one lung and/or
to normalize the end-expiratory impedance values (EELI) over all pixels for at least one lung and to obtain a normalized end-expiratory impedance value (EELI) for each pixel of at least one lung.

31. The device according to claim 19, which is configured to determine and/or display reference data.

32. The device according to claim 31, which is configured to determine and/or display a reference distribution of impedance amplitude values (ΔZ) and fitted values (ΔZ_{ideal}) or a reference histogram on the basis of electrical impedance tomography (EIT) data of reference patients.

33. The device according to claim 19, wherein the calculation and control unit is configured to determine data independently for each of the lungs.

34. The method for determination of a measure for the homogeneity of the lung based on electrical impedance tomography (EIT) data, with a device according to claim 19, the method comprising:
providing EIT data from at least one region of at least one lung (2) of a living being over an observation period (ΔT);
determining impedance values over the observation period for each pixel (1) of the at least one region of at least one lung (2);
determining an impedance amplitude value (ΔZ) for each pixel (1) of at least one lung (2);
determining an end-expiratory impedance value (EELI) for each pixel (1) of at least one lung (2);
associating the impedance amplitude values (ΔZ) and the end-expiratory impedance values (EELI) individually for each pixel (1)
determining data on the basis of the impedance amplitude values (ΔZ) and the associated end-expiratory impedance values (EELI); and
generating a control signal on the basis of the data.

35. The method according to claim 34, comprising comparing the data with a predetermined criterion.

36. The method according to claim 34, comprising a step of predetermining a criterion on the basis of a control group of patients.

37. The method according to claim 34, comprising a step of performing a function fit of the impedance amplitude values (ΔZ) in dependency of the end-expiratory impedance values (EELI) for at least one lung to obtain a fitted function (ΔZ_{ideal}(EELI)), for at least one lung (2) and determining data representative of a distribution of impedance amplitude values (ΔZ) with respect to the fitted values (ΔZ_{ideal}).

38. The method according to claim 37, wherein the fitted function (ΔZ_{ideal}(EELI)) is a linear function ΔZ_{ideal}=αEELI+β.

39. The method according to claim 37, comprising the step of determining for each pixel a deviation value (Strain_{EIT}) representing the deviation of the impedance amplitude value (ΔZ) from the fitted value (ΔZ_{ideal}).

40. The method according to claim 37, wherein the deviation value (Strain_{EIT}) is the ratio (ΔZ/ΔZ_{ideal}) between impedance amplitude values (ΔZ) and the fitted value (ΔZ_{ideal}) at the same end-expiratory impedance value (EELI).

41. The method according to claim 39, comprising the step of determining an amount of pixels for which the deviation value (Strain_{EIT}) is larger than a predetermined threshold and/or for which the deviation value (Strain_{EIT}) is outside a predetermined region.

42. The method according to claim 34, comprising the step of providing or outputting an output signal for displaying a representation of the data.

43. The method according to claim 34, comprising the step of determining data for each of the lungs and/or comprising the step of determining reference data.

44. A computer program product directly loadable into a computer and/or for running on the computer, into a calculation and control unit of a device according to claim 1 and/or an EIT device, wherein the computer program product comprises software code portions for performing the steps of a method comprising:
providing EIT data from at least one region of at least one lung (2) of a living being over an observation period (ΔT);
determining impedance values over the observation period for each pixel (1) of the at least one region of at least one lung (2);
determining an impedance amplitude value (ΔZ) for each pixel (1) of at least one lung (2);
determining an end-expiratory impedance value (EELI) for each pixel (1) of at least one lung (2);
associating the impedance amplitude values (ΔZ) and the end-expiratory impedance values (EELI) individually for each pixel (1)
determining data on the basis of the impedance amplitude values (ΔZ) and the associated end-expiratory impedance values (EELI); and
generating a control signal on the basis of the data.
